(19) United States
(12) Patent Application Publication
(10) Pub. No.: US 2010/0160666 A1
(43) Pub. Date: Jun. 24, 2010

(54) PREPARATION OF GABAPENTIN ENACARBIL INTERMEDIATE

(75) Inventors: Elena BEN MOHA-LERMAN, Kiryat Ono (IL); Anna BALANOV, Rehovot (IL); Miri ADLER, Petach-Tikva (IL)

Correspondence Address: MERCHANT & GOULD PC, P.O. BOX 2903, MINNEAPOLIS, MN, 55402-0903, US

(73) Assignee: TEVA PHARMACEUTICAL INDUSTRIES LTD., Petach-Tikva (IL)

(21) Appl. No.: 12/646,184

(22) Filed: Dec. 23, 2009

Related U.S. Application Data
(60) Provisional application No. 61/203,546, filed on Dec. 23, 2008.

(51) Int. Cl. C07C 227/00; C07C 227/38

(52) U.S. Cl. 560/125

(57) ABSTRACT

Allyl 1 {[(α-isobutanoyloxyethoxy)carbonyl]aminomethyl}-1-cyclohexane acetate can be prepared by combining allyl 1-aminomethyl-1-cyclohexane acetate hydrochloride, a polar organic solvent, chloroethyl chloroformate, and an amine base or an inorganic base selected from a group consisting of carbonate and bicarbonate to provide a reaction mixture; and adding isobutyric acid to the reaction mixture. The product can be purified and/or converted to gabapentin enacarbil.

### CROSS-REFERENCE TO RELATED APPLICATIONS

This application claims the benefit of U.S. Provisional Patent Application Ser. No. 61/203,546, filed Dec. 23, 2008, which is incorporated herein by reference.

### FIELD OF THE INVENTION

The present invention relates to the preparation of allyl 1 {[α-isobutanoyloxyethoxy)carbonyl]aminomethyl}-1-cyclohexane acetate, an intermediate of gabapentin enacarbil, as well as obtaining it with high level of purity, and to its conversion to gabapentin enacarbil.

### BACKGROUND OF THE INVENTION

Gabapentin (GBP), 1-(aminomethyl)cyclohexaneacetic acid is described according to the following formula:

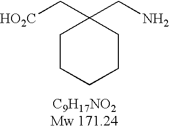

GBP is a white to off-white crystalline solid with a pKal of 3.7 and a pKa2 of 10.7. GBP is marketed by Pfizer under the trade name Neurontin®.

GBP is used in the treatment of cerebral diseases such as epilepsy. In animal models of analgesia, GBP prevents allodynia (pain-related behavior in response to a normally innocuous stimulus) and hyperalgesia (exaggerated response to painful stimuli). GBP also decreases pain related responses after peripheral inflammation. Animal test systems designed to detect anticonvulsant activity, proved that GBP prevents seizures as do other marketed anticonvulsants.

Gabapentin enacarbil (GBPE), 1-{[(α-isobutanoyloxyethoxy)carbonyl]-aminomethyl}-1-cyclohexane acetic acid, is a transported prodrug of GBP and is described according to the following formula:

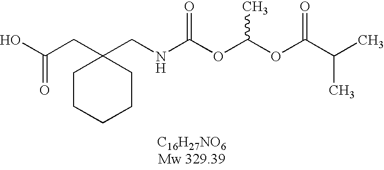

GBPE was developed to improve some of the bioavailability limitations that are known in GBP. GBPE is recognized by high-capacity transport proteins expressed all along the intestinal tract, making it suitable for sustained-release formulation for colonic absorption. After its absorption in the blood, GBPE is rapidly converted to GBP.

GBPE and processes for its preparation are described in U.S. Pat. Nos. 6,818,787; 7,232,924 (“US '924”) and 7,227,028.

US '924 encompasses a process for preparing GBPE as shown in the scheme below:

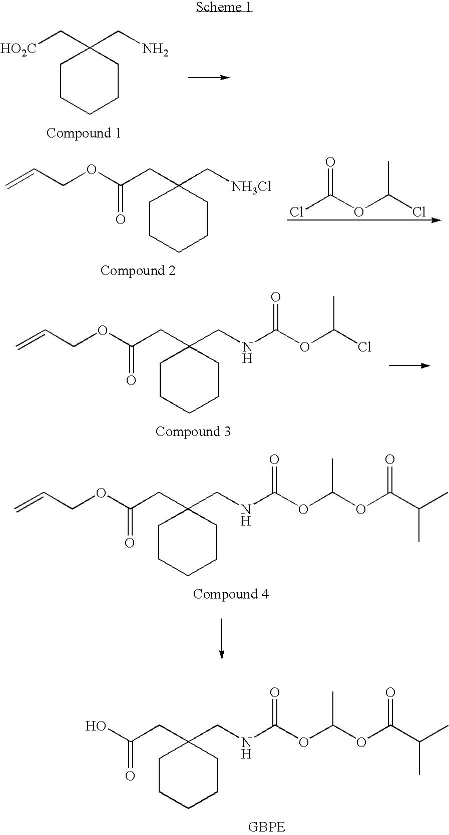

There is a need in the art for improved processes for the preparation of compound 4, as well as obtaining it in high purity.

### SUMMARY OF THE INVENTION

In one embodiment, the present invention encompasses a one pot process for preparing compound 4, an intermediate in the preparation of GBPE, comprising: combining compound 2, a polar organic solvent, chloroethyl chloroformate, and an amine base or an inorganic base selected from a group consisting of: carbonate and bicarbonate to provide a reaction mixture; and adding isobutyric acid to the reaction mixture to obtain compound 4.

In another embodiment, the present invention provides a process for recovering and purifying compound 4 comprising: extracting a reaction mixture containing compound 4 with a C₅-C₁₀ alkane; and removing the C₅-C₁₀ alkane to obtain purified compound 4.

In yet another embodiment, the present invention encompasses a process for preparing GBPE comprising preparing compound 4 by the process described above and further converting it to GBPE.

In one embodiment, in the case when the amine base is Bu₃N, the present invention encompasses a recovery process of Bu₃N comprising adjusting the aqueous phase to a pH of about 8 to about 14; extracting a reaction mixture containing Bu₃N with a water-immiscible organic solvent; and removing the water-immiscible organic solvent to obtain Bu₃N.

### DETAILED DESCRIPTION OF THE INVENTION

The present invention encompasses an improved process for the preparation of GBPE as shown in the scheme below:

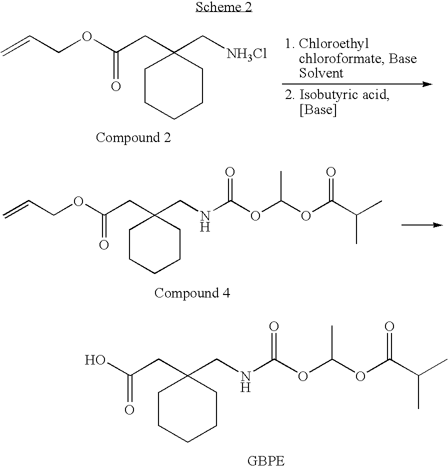

As used herein, the term “compound 2” refers to allyl 1-aminomethyl-1-cyclohexane acetate hydrochloride.

As used herein, the term “compound 3” refers to allyl 1{[α-chloroethoxy)carbonyl]aminomethyl}-1-cyclohexane acetate.

As used herein, the term “compound 4” refers to allyl 1{[α-isobutanoyloxyethoxy)carbonyl]aminomethyl}-1-cyclohexane acetate.

As used herein, the term “GBPE” refers to 1-{[α-isobutanoyloxyethoxy)carbonyl]-aminomethyl}-1-cyclohexane acetic acid.

As used herein, the term “room temperature” refers to a temperature of about 15° C. to about 30° C., typically about 20° C. to about 25° C.

As used herein, the term “ratio” refers to a molar ratio.

As used herein, the term “aromatic hydrocarbon” refers to a compound containing a six-carbon ring containing three double bonds that is normally liquid at about 25° C. Toluene is a preferred aromatic hydrocarbon solvent of the present invention. Other aromatic hydrocarbons useful in the practice of the present invention include benzene, anisole and the xylenes.

As used herein, the term “chlorinated solvent” refers to C₁-C₆ chlorinated hydrocarbon. Preferred chlorinated solvents are selected from the group consisting of: dichloromethane (CH₂Cl₂), dichloroethane, chlorobenzene and chloroform.

As used herein, the term “one-pot” refers to a process done without isolating the process intermediates from the reaction solvent or mixture.

US '924 refers to a process for preparing compound 4 from compound 2 by going through compound 3, as shown in scheme 1. Compound 3 is unstable when exposed to water. Also, going through compound 3 makes the process more complicated. Subsequently, the process of US '924 has a lower yield when compared to the one-pot process described in this invention. The process of the present invention provides compound 4 with high yields while using cheaper and safer solvents, as well as cheaper bases.

In addition, the present invention encompasses a new method for the purification of compound 4 by extracting it from a solvent.

The prior art references, such as US '924, use column chromatography for the purification. Column chromatography is a complicated method, not applicable on an industrial scale and less ecological when compared to the extraction method of the present invention.

GBP used in one embodiment of the present invention may be obtained by any method known in the art, for example, according to U.S. Pat. No. 6,255,526, incorporated herein by a reference.

Compound 2 used in the present invention may be obtained by any method known in the art, for example, according to US '924, incorporated herein by a reference.

The present invention encompasses a one-pot process for preparing compound 4, an intermediate in the preparation of GBPE, comprising: combining compound 2, a polar organic solvent, chloroethyl chloroformate (“CEC”), and an amine base or an inorganic base selected from a group consisting of: carbonate and bicarbonate, to provide a reaction mixture; and adding isobutyric acid to the reaction mixture to obtain compound 4.

Preferably, the process for preparing compound 4 comprises: combining compound 2, a polar organic solvent, and CEC; adding an amine base or an inorganic base selected from a group consisting of: carbonate and bicarbonate, to obtain a reaction mixture; and adding isobutyric acid to the reaction mixture to obtain compound 4.

Preferably, the polar organic solvent is selected from a group consisting of C₃-C₇ ketone, aromatic hydrocarbon, C₆-C₁₀ ether, chlorinated solvent and combinations thereof, more preferably, the polar organic solvent is selected from a group consisting of C₃₋₇ ketone, aromatic hydrocarbon and C₆₋₁₀ ether and combinations thereof, most preferably, the polar organic solvent is selected from a group consisting of aromatic hydrocarbon, C₆₋₁₀ ether and combinations thereof.

Preferably, the polar organic solvent is an aprotic solvent.

Preferably, the C₃-C₇ ketone is selected from a group consisting of methyl isobutyl ketone (“MIBK”), acetone, methyl ethyl ketone and cyclohexanone.

Preferably, the C₆-C₁₀ ether is selected from a group consisting of tetrahydrofuran (“THF”), dioxane, methyl tert-butyl ether (“MTBE”), dimethoxyethane (“glyme”), methyl-THF, diisopropyl ether, diethyl ether and methyl t-butyl ether, more preferably, the C₄-C₈ ether is THF or glyme, most preferably, THF.

Preferably, the chlorinated solvent is selected from a group consisting of chloroform, dichloromethane (“DCM”), dichloroethane and chlorobenzene.

Preferably, the aromatic hydrocarbon is selected from the group consisting of toluene, anisole and xylenes.

Preferably, the solvent is toluene.

Preferably, the CEC to compound 2 ratio is about 1:1 to about 1.5:1, more preferably, about 1.1:1.

Preferably, the amine base is selected from a group consisting of C₅-C₁₂ tertiary amine, C₅-C₁₅ aromatic amine and combinations thereof.

Preferably, the C₅-C₁₂ tertiary amine is selected from a group consisting of N-methylmorpholine (“NMM”), triethyl amine (“TEA”), diisopropyl ethyl amine (“DIPEA”) and tributyl amine (“Bu₃N”).

Preferably, the C₅-C₁₅ aromatic amine is selected from a group consisting of pyridine, 2,6-lutidine, 4-dimethylaminopyridine (“DMAP”) and quinoline.

Preferably, the amine base is selected from a group consisting of NMM, TEA and Bu₃N, more preferably, the base is NMM or Bu₃N, most preferably, the base is Bu₃N.

Preferably, the carbonate is selected from a group consisting of: K₂CO₃, Na₂CO₃ and Cs₂CO₃. Preferably, the bicarbonate is KHCO₃ or NaHCO₃.

Preferably, prior to the base addition, a cooling step is performed. Preferably, the cooling is to a temperature of about 10° C. to about −5° C., more preferably, to about 0° C. Preferably, the cooling is for about 20 minutes to about 40 minutes.

Preferably, the base is added drop-wise. Preferably, the base addition is done for about 20 minutes to about 60 minutes, more preferably, for about 30 minutes.

Preferably, the base to compound 2 ratio is about 3:1 to about 8:1, more preferably, about 4:1 to about 8:1, most preferably, about 7:1.

The base may be added in one portion or in two portions. Preferably, when the first portion of the base to compound 2 ratio is less than 3:1, a second portion of an amine base or an inorganic base selected from a group consisting of: carbonate and bicarbonate is added with the isobutyric acid. Preferably, the base in the second portion is the same as the base in the first portion that is added with compound 2. Optionally, the base in the first portion that is added with compound 2 is in a ratio of about 2:1 or more (base to compound 2), and the ratio of the base in the second portion to compound 2 is about 1:1 or more.

Preferably, following the base addition and prior to the isobutyric acid addition, a stirring step is performed. Preferably, the stirring is for about 1 hour to about 3 hours, more preferably for about 2 hours. Preferably, the stirring is at about room temperature to about 50° C., more preferably, at about room temperature.

Preferably, the isobutyric acid to compound 2 ratio is about 1:1 to about 5:1, more preferably, about 5:1.

Preferably, following the isobutyric acid addition, a stirring step is performed. Preferably, the stirring is for about 12 hours to about 24 hours, more preferably, for about 16 hours. Preferably, the stirring is performed at about room temperature to about 50° C., more preferably, at about room temperature.

Typically, Compound 4 is further recovered and purified. The recovery and purification may be done by a process comprising: extracting a reaction mixture containing compound 4 with C₅-C₁₀ alkane; and removing the C₅-C₁₀ alkane to obtain purified compound 4.

Preferably, the recovery and purification process comprises: adding a C₅-C₁₀ alkane and water to a reaction mixture containing compound 4 to obtain a two-phase system; separating the phases; and removing the C₅-C₁₀ alkane to obtain purified compound 4.

Preferably, the C₅-C₁₀ alkane is hexane. Preferably, the obtained Compound 4 has a purity level of more than about 80% by assay, more preferably, more than about 90% by assay, even more preferably, more than about 95% by assay, and most preferably, more than about 99% by assay, as measured by HPLC. Prior to the removal of the C₅-C₁₀ alkane, the organic phase may be further washed. Preferably, the washing is done with water. Typically, the washing is done a number of times, while repeating the phase separations accordingly. Optionally, the separated organic phase is further washed with NaHCO₃ and brine.

Preferably, prior to the removal of the C₅-C₁₀ alkane, a drying step is performed. Preferably, the drying is done over a salt such as anhydrous Na₂SO₄ or anhydrous MgSO₄. Preferably, the drying is done at about room temperature. Preferably, the removal of the C₅-C₁₀ alkane is done by evaporation.

The obtained aqueous phases may be collected to obtain one aqueous phase. In the case that Bu₃N is used, the Bu₃N may be further recovered from the collected aqueous phase. The present invention encompasses a process for recovering Bu₃N from the collected aqueous phase comprising: adjusting the aqueous phase to a pH of about 8 to about 14; extracting a reaction mixture containing Bu₃N with a water-immiscible organic solvent; and removing the water-immiscible organic solvent to obtain Bu₃N.

Preferably, the recovery process comprises: adjusting the collected aqueous phase to a pH of about 8 to about 14; combining the aqueous phase with water-immiscible organic solvent to obtain a two-phase system; separating the phases; drying the organic phase over Na₂SO₄ or MgSO₄; and evaporating the solvent to obtain Bu₃N. Preferably, the water-immiscible organic solvent is selected from the group consisting of: aromatic solvent such as toluene, C₃-C₇ ester such as ethyl acetate, and C₅-C₁₀ alkane such as hexane, more preferably, the water-immiscible organic solvent is hexane. Preferably, the pH is adjusted to about 8. Preferably, the pH adjustment is done with a base selected from the group consisting of: alkaline base, carbonate and bicarbonate. Preferably, the alkaline base is selected from the group consisting of: NaOH, KOH, LiOH and CsOH. Preferably, the carbonate is selected from the group consisting of: K₂CO₃, Na₂CO₃ and Cs₂CO₃. Preferably, the bicarbonate is KHCO₃ or NaHCO₃. Preferably, the base is NaHCO₃.

Preferably, the yield is about 80%, or more, more preferably, the yield is about 80% to about 95%, most preferably, the yield is about 80% to about 95%, or more, by weight.

The recovery of the Bu₃N provides an ecological, commercial and financial advantage, since it reduces the amount of the discarded base and the base may be used again.

The present invention also encompasses a process for preparing GBPE comprising preparing compound 4 by the process described above and further converting it to GBPE.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. Absent statement to the contrary, any combination of the specific embodiments described above are consistent with and encompassed by the present invention.

### Instruments:

HPLC method:

| Column & Packing: | Acentis Express 150 × 4.6 2.7μ | | |
|---|---|---|---|
| Eluent A: | A: 0.05% TFA in water | | |
| Eluent B: | B: 0.05% TFA in Acetonitrile | | |
| | Time | % Eluent A | % Eluent B |
| Gradient | 0 | 75 | 25 |
| | 5 | 75 | 25 |
| | 25 | 10 | 90 |

| | |
|---|---|
| Stop time: | 50 min |
| Equilibrium time: | 8 min |
| Flow: | 1.0 ml/min |
| Sample volume: | 10 μL |
| Detector: | Corona |
| Column temperature: | 25° C. |
| Diluent | Water:ACN (50:50) |

### Sample Preparation

Weigh accurately about 50 mg of sample in a 10 ml amber volumetric flask. Dissolve with diluent.

### Method

Inject the sample solutions into the chromatograph, continuing the chromatogram of sample up to the end of the gradient. Determine the areas for each peak in each solution using a suitable integrator.

### Calculation

The calculation for assay and impurity content was performed as follows:

$$Content ( \% ) = \frac{{A}_{sample} * {C}_{std} * {P}_{std}}{{A}_{std} * {C}_{sample}}$$

Where Aₛₐₘₚₗₑ is the area of the peak of required component in the chromatogram of the sample solution. A_{std} is the average area of the peak compound 4 in the chromatograms of the standard solution. Cₛₐₘₚₗₑ is the concentration of the sample solution (g/ml). C_{std} is the concentration of compound 4 in the standard solution (g/ml). P_{std} is the purity of the standard (%).

### EXAMPLES

### Example 1

### Preparation of Allyl-GBPE

Compound 2 (1 g, 4.04 mmol) was added to Solvent followed by addition of Chloroethyl chloroformate (0.48 ml, 4.44 mmol). The flask was cooled in ice-water bath followed by dropwise addition of Base A over a period of 30 minutes. When the addition was finished the reaction was allowed to reach room temperature and was stirred at this temperature for additional 2 hours. Isobutyric acid (1.8 ml, 20.2 mmol) was added to the reaction mixture followed by dropwise addition of premixed solution of Isobutyric acid (1.8 ml, 20.2 mmol) and Base B. The reaction was stirred at room temperature for 16 h and then diluted with Hexane and water. The phases were separated, and the organic phase was washed twice with water, twice with NaHCO₃ and brine. The organic phase was dried over anhydrous Na₂SO₄ and evaporated to give the desired product.

The experiments are summarized in the following table:

| Solvent | | Base A | | | Base B | | | | |
|---|---|---|---|---|---|---|---|---|---|
| Type | Volume | Type | m | n | Type | m | n | Yield | Assay |
| Toluene | 3 ml | NMM | 0.9 g | 8.9 mmol | NMM | 2 g | 20.2 mmol | 91.80% | 100% |
| THF | 3 ml | NMM | 0.9 g | 8.9 mmol | NMM | 2 g | 20.2 mmol | 91% | 100% |
| Glyme | 3 ml | NMM | 0.9 g | 8.9 mmol | NMM | 2 g | 20.2 mmol | 86.50% | 93% |
| DCM | 3 ml | TEA | 0.9 g | 8.9 mmol | TEA | 2 g | 20.2 mmol | 88.50% | 87% |
| Toluene | 3 ml | TEA | 0.9 g | 8.9 mmol | TEA | 2 g | 20.2 mmol | 80.16% | 77% |
| Toluene | 3 ml | Bu₃N | 1.64 g | 8.9 mmol | Bu₃N | 3.7 g | 20.2 mmol | 92.00% | 100% |
| Glyme | 3 ml | TEA | 0.9 g | 8.9 mmol | TEA | 2 g | 20.2 mmol | 86.50% | 85.5% |

### Example 1a

### Recovery of the Bu₃N

Following the procedure above, when Bu₃N is used, the combined aqueous phase from the previous work-up was basified with solid NaHCO₃ to pH=8 and extracted twice with hexane. The organic phase was dried over anhydrous Na₂SO₄ and evaporated to give Bu₃N up to 95% total recovery.

1. A one-pot process for preparing allyl 1 {[(α-isobutanoyloxyethoxy)carbonyl]aminomethyl}-1-cyclohexane acetate (compound 4) comprising: combining allyl 1-aminomethyl-1-cyclohexane acetate hydrochloride (compound 2), a polar organic solvent, chloroethyl chloroformate (“CEC”), and an amine base or an inorganic base selected from a group consisting of: carbonate and bicarbonate to provide a reaction mixture; and adding isobutyric acid to the reaction mixture to obtain allyl 1 {[α-isobutanoyloxyethoxy)carbonyl]aminomethyl}-1-cyclohexane acetate (compound 4).

2. The process of claim 1, wherein the process comprises: combining allyl 1-aminomethyl-1-cyclohexane acetate hydrochloride (compound 2), a polar organic solvent, and chloroethyl chloroformate; adding an amine base or an inorganic base selected from a group consisting of: carbonate and bicarbonate to obtain a reaction mixture; and adding isobutyric acid to the reaction mixture to obtain allyl 1 {[α-isobutanoyloxyethoxy)carbonyl]aminomethyl}-1-cyclohexane acetate (compound 4).

3. The process of claim 1, wherein the polar organic solvent is selected from a group consisting of: C₃-C₇ ketone, aromatic hydrocarbon, C₆-C₁₀ ether, chlorinated solvent and combinations thereof.

4. The process of claim 1, wherein the polar organic solvent is selected from a group consisting of: C₃-C₇ ketone, aromatic hydrocarbon, C₆-C₁₀ ether and combinations thereof.

5. The process of claim 4, wherein the polar organic solvent is selected from a group consisting of: aromatic hydrocarbon, C₆-C₁₀ ether and combinations thereof.

6. The process of claim 1, wherein the polar organic solvent is selected from a group consisting of: methyl isobutyl ketone (“MIBK”), methyl ethyl ketone, cyclohexanone and acetone.

7. The process of claim 1, wherein the polar organic solvent is selected from a group consisting of: tetrahydrofuran (“THF”), dioxane, methyl tert-butyl ether (“MTBE”), dimethoxyethane (“glyme”), methyl-THF, diisopropyl ether, diethyl ether and methyl t-butyl ether.

8. The process of claim 7, wherein the polar organic solvent is THF or glyme.

9. The process of claim 8, wherein the solvent is THF.

10. The process of claim 1, wherein the polar organic solvent is selected from a group consisting of: chloroform, dichloromethane (“DCM”), dichloroethane and chlorobenzene.

11. The process of claim 1, wherein the polar organic solvent is selected from the group consisting of: toluene, anisole and xylenes.

12. The process of claim 11, wherein the solvent is toluene.

13. The process of claim 1, wherein the CEC to allyl 1-aminomethyl-1-cyclohexane acetate hydrochloride (compound 2) ratio is about 1:1 to about 1.5:1.

14. The process of claim 13, wherein the CEC to allyl 1-aminomethyl-1-cyclohexane acetate hydrochloride (compound 2) ratio is about 1.1:1.

15. The process of claim 1, wherein the reaction mixture comprises an amine base selected from a group consisting of: C₅-C₁₂ tertiary amine, C₅-C₁₅ aromatic amine and combinations thereof.

16. The process of claim 15, wherein the amine base is selected from a group consisting of: N-methylmorpholine (“NMM”), triethyl amine (“TEA”), diisopropyl ethyl amine (“DIPEA”), tributyl amine (“Bu₃N”), pyridine, 2,6-lutidine, 4-dimethylaminopyridine (“DMAP”) and quinoline.

17. The process of claim 16, wherein the amine base is selected from a group consisting of: NMM, TEA and Bu₃N.

18. The process of claim 17, wherein the amine base is NMM or Bu₃N.

19. The process of claim 18, wherein the amine base is Bu₃N.

20. The process of claim 1, wherein the reaction mixture comprises a carbonate selected from a group consisting of: K₂CO₃, Na₂CO₃ and Cs₂CO₃.

21. The process of claim 1, wherein the reaction mixture comprises KHCO₃ or NaHCO₃.

22. The process of claim 1, wherein the base to allyl 1-aminomethyl-1-cyclohexane acetate hydrochloride (compound 2) ratio is about 3:1 to about 8:1.

23. The process of claim 22, wherein the base to allyl 1-aminomethyl-1-cyclohexane acetate hydrochloride (compound 2) ratio is about 4:1 to about 8:1.

24. The process of claim 23, wherein the base to allyl 1-aminomethyl-1-cyclohexane acetate hydrochloride (compound 2) ratio is about 7:1.

25. The process of claim 1, wherein the base is added in two portions and the ratio in the first portion of the base to allyl 1-aminomethyl-1-cyclohexane acetate hydrochloride (compound 2) is less than 3:1.

26. The process of claim 25, wherein the second portion of the base is added with the isobutyric acid.

27. The process of claim 26, wherein the base in the second portion is the same as the base in the first portion, and wherein the first portion is added with allyl 1-aminomethyl-1-cyclohexane acetate hydrochloride (compound 2).

28. The process of claim 26, wherein the base in the first portion is added with allyl 1-aminomethyl-1-cyclohexane acetate hydrochloride (compound 2), and wherein the ratio of the base in the first portion to allyl 1-aminomethyl-1-cyclohexane acetate hydrochloride (compound 2) is about 2:1 or more.

29. The process of claim 28, wherein ratio of the base in the second portion to allyl 1-aminomethyl-1-cyclohexane acetate hydrochloride (compound 2) is about 1:1 or more.

30. The process of claim 1, wherein the isobutyric acid to allyl 1-aminomethyl-1-cyclohexane acetate hydrochloride (compound 2) ratio is about 1:1 to about 5:1.

31. The process of claim 30, wherein the isobutyric acid to allyl 1-aminomethyl-1-cyclohexane acetate hydrochloride (compound 2) ratio is about 5:1.

32. A process for recovering and purifying allyl 1 {[α-isobutanoyloxyethoxy)carbonyl]aminomethyl}-1-cyclohexane acetate (compound 4) comprising: extracting a reaction mixture containing allyl 1 {[α-isobutanoyloxyethoxy)carbonyl]aminomethyl}-1-cyclohexane acetate (compound 4) with a C5-C10 alkane; and removing the C5-C10 alkane to obtain purified allyl 1 {[α-isobutanoyloxyethoxy)carbonyl]aminomethyl}-1-cyclohexane acetate (compound 4).

33.-46. (canceled)
